(19) United States
(12) Patent Application Publication
(10) Pub. No.: US 2026/0137353 A1
(43) Pub. Date: May 21, 2026

(54) METHOD TO DETERMINE INSULIN RESISTANCE FROM CONTINUOUS GLUCOSE MONITORING (CGM) FOR PRE-DIABETIC CONDITIONS

(71) Applicant: METADVICE (SUISSE) SA, St-Sulpice (CH)

(72) Inventors: André Bernhard Jaun, Bremblens (CH); Arina Lozhkina, Lausanne (CH)

(21) Appl. No.: 19/391,444

(22) Filed: Nov. 17, 2025

Related U.S. Application Data
(60) Provisional application No. 63/721,017, filed on Nov. 15, 2024.

(51) Int. Cl. A61B 5/00; A61B 5/145; A61B 5/1495

(52) U.S. Cl. CPC A61B 5/7275; A61B 5/1451; A61B 5/14532; A61B 5/1495; A61B 2560/0223

(57) ABSTRACT

Methods of predicting insulin resistance in a subject are provided. The methods include measuring interstitial fluid glucose in the subject over a period of time to obtain a plurality of glucose values (G(tₙ)); fitting the measured plurality of glucose values (G(tₙ)) to a glucose-insulin response model to calculate a plurality of subject-specific parameters, the glucose-insulin response model comprising a modified Lotka-Volterra system of equations for the glucose-insulin response pair (G(t), I(t)); and comparing at least one of the calculated subject-specific parameters to a set threshold value to predict insulin resistance in the subject. Systems for predicting insulin resistance in a subject are also provided. The systems include a continuous glucose monitoring (CGM) sensor and a computation device.

### CROSS-REFERENCE TO RELATED APPLICATION

This application claims priority under 35 U.S.C. § 119(e) to U.S. Provisional Application No. 63/721,017 filed Nov. 15, 2024 and titled “METHOD TO DETERMINE INSULIN RESISTANCE FROM CONTINUOUS GLUCOSE MONITORING (CGM) FOR PRE-DIABETIC CONDITIONS,” which is incorporated herein by reference in its entirety for all purposes.

### FIELD OF TECHNOLOGY

Aspects and embodiments disclosed herein are generally related to determining insulin resistance from continuous glucose monitoring, and more specifically, to systems and methods for predicting insulin resistance from calculated insulin response values.

### SUMMARY

In accordance with an aspect, there is provided a method of predicting insulin resistance in a subject. The method may comprise measuring interstitial fluid glucose in the subject over a period of time to obtain a plurality of glucose values (G(tₙ)); fitting the measured plurality of glucose values (G(tₙ)) to a glucose-insulin response model to calculate a plurality of subject-specific parameters, the glucose-insulin response model comprising a modified Lotka-Volterra system of equations for the glucose-insulin response pair (G(t), I(t)); and comparing at least one of the calculated subject-specific parameters to a set threshold value to predict insulin resistance in the subject.

In some embodiments, the method may further comprise calibrating the predicted insulin resistance in the subject against a HOMA-IR homeostatic model assessment of insulin resistance to confirm the prediction of insulin resistance in the subject.

In some embodiments, a HOMA-IR≤2 is negative for insulin resistance and a HOMA-IR>2 is positive for insulin resistance.

In some embodiments, the method may further comprise generating the glucose-insulin response model from an initial measurement of glucose (G(t₀)) and an arbitrarily chosen value for insulin (I₀).

In some embodiments, the plurality of subject-specific parameters are selected from rate of glucose uptake by insulin-dependent tissues (K_{g}), net balance between hepatic glucose output and insulin-independent glucose uptake by brain (T_{g}), apparent first-order disappearance rate constant for insulin (Kᵢ), and endogenous insulin production rate in the presence of glucose (Vᵢ).

In some embodiments, the plurality of subject-specific parameters includes the rate of glucose uptake by insulin-dependent tissues (K_{g}) and the threshold value is set as κ, with Kᵢ>κ being positive for insulin resistance.

In some embodiments, the plurality of subject-specific parameters includes the insulin-independent glucose uptake by brain (T_{g}) and the threshold value s set as τ, with T_{g}>τ being positive for insulin resistance.

In some embodiments, the interstitial fluid glucose of the subject is measured with a continuous glucose monitoring (CGM) sensor.

In some embodiments, the period of time is at least 2 weeks.

In some embodiments, the method may further include providing the CGM sensor.

In some embodiments, the method may comprise measuring the interstitial fluid glucose every 0.25-5 minutes to obtain the plurality of glucose values.

In some embodiments, the method may comprise measuring the interstitial fluid glucose at irregular intervals over the period of time.

In some embodiments, the subject is non-diabetic or pre-diabetic.

In some embodiments, the subject is unmedicated for a metabolic disorder.

In some embodiments, the method may further include treating the subject for a metabolic disorder, metabolic imbalance or disturbance, or symptom thereof in response to a positive predicted insulin resistance.

In accordance with another aspect, there is provided a system for predicting insulin resistance in a subject. The system may comprise a continuous glucose monitoring (CGM) sensor having an analyzer configured to measure glucose data from interstitial fluid of the subject and a transmitter configured to transmit the glucose data. The system may comprise a computation device comprising a receiver configured to receive the glucose data, the computation device being programmed to fit the measured glucose data from the subject to a glucose-insulin response model to calculate a plurality of subject-specific parameters and compare at least one of the calculated subject-specific parameters to a set threshold value to predict insulin resistance in the subject, the glucose-insulin response model comprising a modified Lotka-Volterra system of equations for the glucose-insulin response pair (G(t), I(t)).

In some embodiments, the computation device is further programmed to calibrate the predicted insulin resistance against a HOMA-IR homeostatic model assessment of insulin resistance to confirm the prediction of insulin resistance in the subject.

In some embodiments, the computation device is further programmed to generate the glucose-insulin response model from an initial measurement of glucose (G(t₀)) and an arbitrarily chosen value for insulin (I₀).

In some embodiments, the CGM sensor comprises a power source capable of powering the CGM sensor for at least 1 week.

In some embodiments, the CGM sensor comprises a power source capable of powering the CGM sensor for at least 2 weeks.

In some embodiments, the CGM sensor comprises a power source capable of powering the CGM sensor for at least 4 weeks.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings are not drawn to scale. In the drawings, each identical or nearly identical component that is illustrated in the various figures is represented by a like numeral. For purposes of clarity, not every component may be labeled in every drawing. In the drawings:

FIG. 1 includes graphs showing continuous glucose monitoring traces for a healthy person (top) and a type-2 diabetic subject (bottom);

FIG. 2 includes graphs showing glucose data fitted with an RMSE of 0.85 (top) and insulin generated (bottom) for a healthy individual;

FIG. 3 is a graph showing the fitting results for two parameters Kᵢ and T_{g} over a 7-day window. The color indicates subject phenotypes, with green for type 2 diabetes, blue for obese and prediabetic, and yellow for healthy individuals. A cross indicates HOMA-IR≤2, and a point indicates HOMA-IR>2; and

FIG. 4 is a box diagram of a system for predicting insulin resistance in a subject, according to one embodiment.

### DETAILED DESCRIPTION

Insulin resistance plays an important role in metabolic disorders. The Homeostatic Model Assessment for Insulin Resistance (HOMA-IR), a widely used measure of insulin resistance, requires quantification of fasting glucose and insulin, limiting its use in clinical settings. Continuous glucose monitoring (CGM) systems allow insights into glucose levels at minimal burden, yet with standard time-in-range and average-based metrics, detailed insights into glucose-insulin homeostasis are lacking.

Insulin resistance in type-2 diabetes generally develops over time, often years before a formal diagnosis can be made. Because of the difficulty of measuring fasting glucose and insulin in controlled clinical settings, the HOMA-IR index that provides a direct measure is rarely available to guide pre-diabetic subjects. This disclosure shows how home readings from continuous glucose monitoring (CGM) sensors can be used as a more widely available surrogate predictor of insulin resistance. Specifically, the methods disclosed herein may be used to estimate insulin resistance based on free living GCM only, offering an accessible surrogate marker.

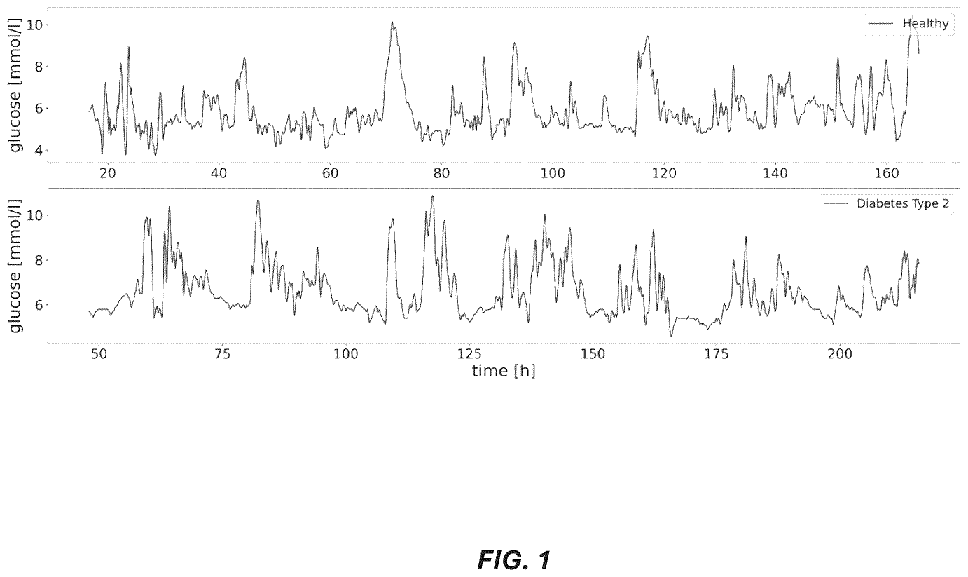

While continuous glucose monitoring is an efficient tool to monitor glucose levels, for type-2 diabetes subjects, CGM alone does not provide complete information about the subject's condition due to complex insulin-glucose dynamics and varying glycemic impacts, which are influenced by factors such as diet, physical activity, hormone levels, and stress. Furthermore, interpreting CGM data under uncontrolled conditions can be challenging. As an example, FIG. 1 shows the CGM readings over a week for two subjects: one healthy and one type-2 diabetic. As seen in FIG. 1, there was little difference in the two CGM readings, even if the HOMA-IR index, quantified only once by measuring blood insulin and glucose during fasting conditions, showed a significant difference. The healthy subject had a HOMA-IR of 1.23, while the diabetic subject had a HOMA-IR of 6.6.

The difference in measured glucose levels between the two subjects reflected a non-linear interplay with unobserved insulin that cannot be observed using representations of CGM data such as that of FIG. 1. This complex interplay can be modeled with Lotka-Volterra, i.e., predator-prey, differential equations, with subject-specific parameters that are a priori unknown but can be computed to fit the high-resolution CGM data.

As disclosed herein, there are provided methods for predicting insulin resistance, e.g., from measured CGM data. The methods may include measuring interstitial fluid glucose in a subject over an extended period of time and fitting the measured glucose values to a glucose-insulin response model.

Continuous glucose monitoring data may be obtained by a CGM sensor worn by the subject. A CGM sensor is a small device that continuously measures glucose levels in interstitial fluid and transmits glucose data to a control module. The CGM sensor may contain a small analyzer configured to be deployed just beneath the skin, in contact with interstitial fluid of the patent. The CGM sensor may include a transmitter that sends data, usually wirelessly, to a receiver such as a computing device, running a user interface which displays glucose values as a list, graph, or trendline. CGM sensors may be used by individuals with or without diabetes to track glucose trends and manage blood sugar more effectively.

Continuous glucose measurements may be obtained and transmitted every 0.25-5 minutes, for example, every 0.25-1 min, 1-3 min, or 3-5 min, at regular or irregular intervals. The CGM data may be collected over a period of time. In certain embodiments, CGM data may be collected over a period of at least 1 week, at least 2 weeks, at least 3 weeks, or at least 4 weeks.

The subject may include an animal, a mammal, a human, or a non-human animal. In certain embodiments, the subject is a mammalian subject, and in particular embodiments, the subject is a human subject. Although applications with humans are clearly foreseen, veterinary applications, for example, with non-human animals, are also envisaged herein. In certain embodiments, the subject may be non-diabetic or pre-diabetic. Thus, the subject may be unmedicated for a metabolic disorder, metabolic imbalance or disturbance, or symptom thereof, e.g., diabetes, for example, the subject may be free of any medication that alters or controls blood glucose levels and/or absorption of glucose. In other embodiments, the subject may be diagnosed with a metabolic disorder, metabolic imbalance or disturbance, or symptom thereof, e.g., the subject may be diabetic. The subject may be medicated for a metabolic disorder, e.g., diabetes, for example, the subject may receive medication that controls blood glucose levels and/or alters absorption of glucose.

The methods may include fitting the measured glucose values to a glucose-insulin response model. Relying on the high frequency of CGM measurements, a non-linear fit of a modified Lotka-Volterra glucose-insulin response model may be used to reproduce the glucose evolution by estimating the rate of glucose uptake from insulin-dependent tissues and the net hepatic output in excess of the glucose directly absorbed from meals. Utilizing a volume of discontinuous glucose measurements G(tₙ) obtained at irregular times tₙ, the prediction calculation may begin by identifying a finite number of peaks G^{max}(tₖ) that correspond to meals.

Each identified peak results in a source of glucose that may be modeled with a skewed shaping function Mₐ(x, w)=2/w φ(x) Φ(α x), where x=(t−tₖ)/w is defined as the normalized time, α as the skew, w as a width, and φ and Φ are the cumulative and normal distributions, respectively. The rate of glucose appearance R_{α}(t, w) may be modeled as a superposition of peaks where the amplitudes uₖ are calculated from quadratures corresponding to the total amount of glucose intake for each meal.

A Lotka-Volterra system of equations may be used to fit a dynamical evolution of the glucose-insulin response pair (G(t), I(t)), starting from initial conditions that are established from the initial measurement of glucose G(t₀) and an arbitrarily chosen value for insulin I₀, modified by food intake R_{α}(t, w) and a constant source of insulin Rᵢ₀, generating the glucose-insulin response model:

$$dG / dt = - ( {K}_{g} \times G \times I ) + {T}_{g} + {R}_{α} ( t , w ) \qquad Eq . 1$$

$$dI / dt = - ( {K}_{i} \times I ) + {V}_{i} \times G + {R}_{i 0}$$

$$with {R}_{α} ( t , w ) = {\sum }_{k} {u}_{k} {M}_{α} ( ( t - {t}_{k} ) / w , w )$$

A priori unknown parameters of the glucose-insulin response model that characterize each individual subject include the rate of glucose uptake by insulin-dependent tissues K_{g}, the net balance between hepatic glucose output and insulin-independent glucose uptake by brain T_{g}, the apparent first-order disappearance rate constant for insulin Kᵢ, and the endogenous insulin production rate in the presence of glucose Vᵢ. Thus, the glucose-insulin response model may be individualized for each subject.

The methods may include calculating a plurality of subject-specific parameters, for example, one or more of K_{g}, T_{g}, Kᵢ, Vᵢ. Those parameters may be calculated using a non-linear Gauss-Newton iteration with a centered finite difference approximation for the Jacobian and by minimizing the root mean square error (RMSE) between the calculated and the measured glucose levels Σₙ∥G(tₙ)−G(t)∥².

The methods may include comparing the plurality of subject-specific parameters to set thresholds to predict insulin resistance. Using the calculated subject-specific parameters, e.g., Kᵢ and T_{g}, as synthetic biomarkers that signal a reduced level of control for insulin to steer the evolution of glucose levels, insulin resistance may be identified when glucose levels evolve largely independently of the insulin, for instance when Kᵢ>κ and/or T_{g}>τ, with κ and τ being threshold values for insulin resistance that were calibrated from prior studies. In exemplary embodiments, κ may be set to 0.83 and τ may be set to 1.64. These exemplary values were calibrated based on a study involving healthy and pre-diabetic patients under non-therapeutic conditions. Alternate values for κ and τ may be calculated for different conditions.

The methods may further include calibrating the predicted glucose-insulin response against a HOMA-IR homeostatic model assessment to confirm the prediction of insulin resistance in the subject. Under this framework, insulin resistance predictions may be compared with independent measurements, e.g., the HOMA-IR index, which may be used as a binary classifier of insulin resistance status, with HOMA-IR≤2 being negative for insulin resistance and HOMA-IR>2 being positive for insulin resistance. HOMA-IR is calculated using the fasting plasma glucose and insulin concentration:

$$HOMA - IR = \frac{[ fasting glucose ] \times [ fasting insulin ]}{( 22.5 \times 6 )} \qquad Eq . 2$$

where the fasting glucose concentration is in mM and the fasting insulin concentration is in pM. Thus, in certain embodiments, the methods may include obtaining fasting glucose and insulin concentrations of the subject. The methods may include collecting at least one fasted blood sample from the subject and measuring glucose and insulin. The HOMA-IR index depends upon both peripheral and hepatic insulin sensitivity, the contribution of which differs between subjects with normal and elevated fasting glucose concentrations.

Responsive to a positive prediction of insulin resistance, the methods may include diagnosing the subject for a metabolic disorder, metabolic imbalance or disturbance, or symptom thereof, or diagnosing the patient as trending toward the metabolic disorder, metabolic imbalance, disturbance, or symptom thereof. Metabolic disorders are pathological conditions characterized by impairments in the regulation or utilization of glucose and insulin, disrupting the biochemical pathways responsible for cellular energy production and macromolecular synthesis. Exemplary metabolic disorders, imbalances or disturbances, and symptoms include Type 1 and Type 2 diabetes, metabolic syndrome, hypoglycemia, hyperglycemia, thyroid disorders, such as hypothyroidism or hyperthyroidism, and others. A patient who is diagnosed as trending toward the metabolic disorder, metabolic imbalance, disturbance, or symptom thereof may be diagnosed as being pre-diabetic.

Responsive to the positive prediction of insulin resistance, in certain embodiments, the methods may further include treating the subject for a metabolic disorder, metabolic imbalance or disturbance, or symptom thereof. The treatment may include any treatment approved for management of the metabolic disorder, imbalance or disturbance, or symptom, e.g., administering medication that alters or controls blood glucose levels and/or absorption of glucose, recommending lifestyle changes, such as diet and exercise changes, and other approved treatments.

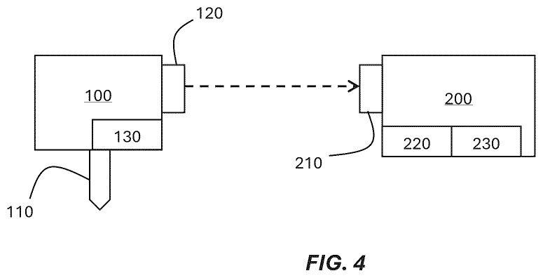

In accordance with another aspect, there is provided a system for predicting insulin resistance in a subject. FIG. 4 is a box diagram of an exemplary system. The system may include a continuous glucose monitoring (CGM) sensor 100 configured for extended use. For instance, the CGM sensor 100 may have a power source 130 capable of powering the sensor 100 for at least 1 week, at least 2 weeks, at least 3 weeks, at least 4 weeks or more, while taking continuous measurements and transmitting data. The CGM sensor 100 may have an analyzer 110 configured to measure glucose data from interstitial fluid of the subject and a transmitter 120 configured to transmit the glucose data.

The system may include a computation device 200 comprising a receiver 210 configured to receive the glucose data from the CGM sensor transmitter 120. The computation device 200 may comprise a processor 220 and a memory storing data 230, and be programmed to execute the functions described herein to predict insulin resistance in the subject. In certain embodiments, the computation device 200 may be programmed to generate tables, graphs, charts, etc. displaying collected and/or generated data.

The computation device 200 may comprise, for example, a mobile device, such as a tablet, cell phone, laptop, or other computer, in communication with the sensor 100. Thus, in certain embodiments, the sensor 100 and computation device 200 may be connectable to each other via a network, such as a local area network or Bluetooth® network, or other network, and programmed to receive and/or transmit data via the network.

### Example: Insulin Resistance Prediction

In a non-limiting example, the methods disclosed herein were tested using three study groups including 34 subjects: 15 healthy, 16 obese pre-diabetic, and three type-2 diabetic subjects. The pre-diabetic subjects' measurements were obtained during diet studies: seven subjects were on a reduced carbohydrate diet and the remaining nine subjects were on a standard reduced calorie diet.

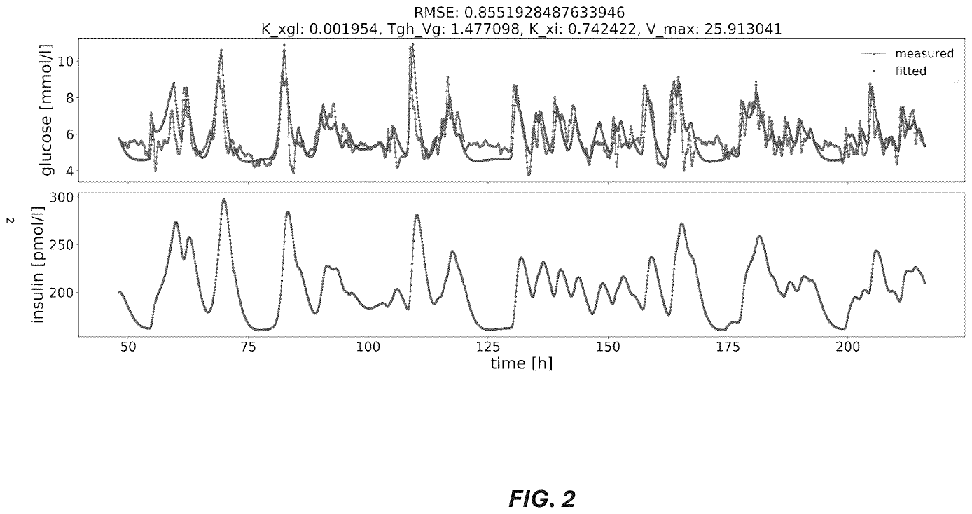

The calculated HOMA-IR was >2 in 20 of the subjects and ≤2 in 14 subjects. An example of a calculated fit to the measured CGM data is illustrated in FIG. 2. The inclusion criteria for the modeling were an RMSE of less than 1.3 for all windows of the subject's measurements and the presence of fasting glucose and insulin data to calculate HOMA-IR. The threshold for insulin resistance was defined as HOMA-IR>2. Comparison between the HOMA-IR and phenotypes for the pre-diabetic subject population demonstrated that three pre-diabetic subjects had a HOMA-IR less than 2 while all other pre-diabetic subjects had a HOMA-IR greater than 4.3. All type-2 diabetic subjects had a HOMA-IR greater than 2.5. Healthy subjects typically have a HOMA-IR ranging from 1.3 to 2.2. One subject labeled healthy had a HOMA-IR of 11.

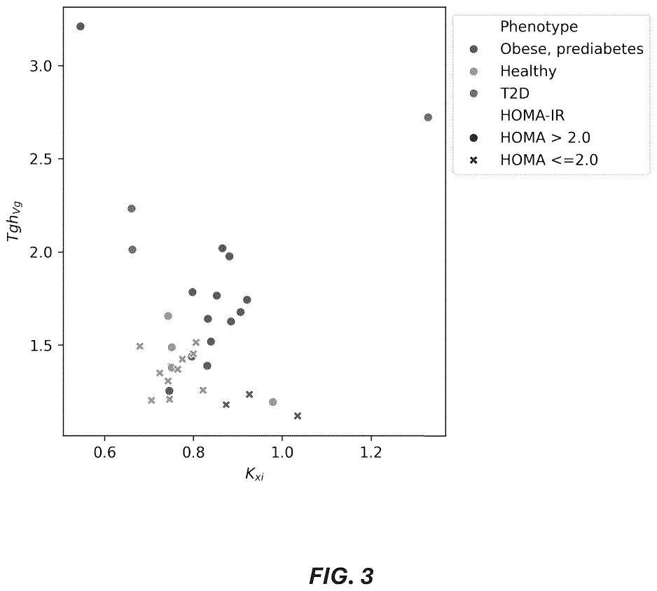

A fitting window of seven days was selected to capture the increase and decrease of glucose under various conditions. This fitting window balanced the standard deviation of coefficients during the full available period of CGM measurements and the maximum RMSE among the fitting windows. FIG. 3 illustrates the fitting results for Kᵢ and T_{g} over a 7-day window for various subject phenotypes. As illustrated in FIG. 3, the approximate thresholds for two Kᵢ and T_{g} were identified as Kᵢ of 0.83 min⁻¹ and T_{g} of 1.64 mM*min⁻¹. FIG. 3 illustrates the formation in the dataset into two groups: healthy individuals and those with obesity or type-2 diabetes. These thresholds were used to classify individuals as either insulin resistant or non-insulin resistant.

To evaluate the effectiveness of these thresholds, the classification results were compared with the calculated HOMA-IR. Using these thresholds, an accuracy of 80% (27/34) was achieved, with a sensitivity of 95% (19/20) and a specificity of 57% (8/14) in detecting insulin resistance when compared to the HOMA-IR values. The CGM data for the subjects shown in FIG. 1 were well separated. The healthy subjects had an average Kᵢ of 0.7 min⁻¹ and average T_{g} of 1.2 mM*min⁻¹, and the diabetic subjects had an average Kᵢ of 0.66 min⁻¹ and average T_{g} of 2.23 mM*min⁻¹. The phenotypes were evaluated, with an accuracy of 88% (30/34).

While it was noted that there was distinction among insulin-resistant and healthy individuals, it was also observed that obese individuals with HOMA-IR indices below 2 could be differentiated from those with a HOMA-IR index exceeding 4.3. In the obese pre-diabetic subjects, the HOMA-IR indices increased with certain parameter thresholds. For example, in the obese pre-diabetic subjects when Kᵢ>0.8 min⁻¹ and T_{g}>1.8 mM*min⁻¹, the calculated HOMA-IR indices exceeded 7.6. Conversely, when these parameters are below the calculated thresholds, the HOMA-IR indices did not exceed 6.8. For obese individuals, the Spearman correlation between the combined parameters and HOMA-IR was 0.758, indicating a strong relationship.

The data suggest that CGM alone may allow prediction of insulin resistance. Moreover, the combination of insulin-glucose dynamics parameters can be applied not only in detecting insulin resistance risks but also in evaluating the severity of these risks. An advantage of the methods disclosed herein is that it was surprisingly discovered that two weeks of CGM measurements are sufficient for accurately predicting insulin resistance in an individual in an uncontrolled setting.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated that various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

What is claimed is:

1. A method of predicting insulin resistance in a subject, the method comprising:
measuring interstitial fluid glucose in the subject over a period of time to obtain a plurality of glucose values (G(tₙ));
fitting the measured plurality of glucose values (G(tₙ)) to a glucose-insulin response model to calculate a plurality of subject-specific parameters, the glucose-insulin response model comprising a modified Lotka-Volterra system of equations for the glucose-insulin response pair (G(t), I(t)); and
comparing at least one of the calculated subject-specific parameters to a set threshold value to predict insulin resistance in the subject.

2. The method of claim 1, further comprising calibrating the predicted insulin resistance in the subject against a HOMA-IR homeostatic model assessment of insulin resistance to confirm the prediction of insulin resistance in the subject.

3. The method of claim 2, wherein a HOMA-IR≤2 is negative for insulin resistance and a HOMA-IR>2 is positive for insulin resistance.

4. The method of claim 1, further comprising generating the glucose-insulin response model from an initial measurement of glucose (G(t₀)) and an arbitrarily chosen value for insulin (I₀).

5. The method of claim 1, wherein the plurality of subject-specific parameters are selected from rate of glucose uptake by insulin-dependent tissues (K_{g}), net balance between hepatic glucose output and insulin-independent glucose uptake by brain (T_{g}), apparent first-order disappearance rate constant for insulin (Kᵢ), and endogenous insulin production rate in the presence of glucose (Vᵢ).

6. The method of claim 5, wherein the plurality of subject-specific parameters includes the rate of glucose uptake by insulin-dependent tissues (K_{g}) and the threshold value is set as κ, with Kᵢ>κ being positive for insulin resistance.

7. The method of claim 5, wherein the plurality of subject-specific parameters includes the insulin-independent glucose uptake by brain (T_{g}) and the threshold value is set as τ, with T_{g}>τ being positive for insulin resistance.

8. The method of claim 1, wherein the interstitial fluid glucose of the subject is measured with a continuous glucose monitoring (CGM) sensor.

9. The method of claim 8, wherein the period of time is at least 2 weeks.

10. The method of claim 8, further including providing the CGM sensor.

11. The method of claim 8, comprising measuring the interstitial fluid glucose every 0.25-5 minutes to obtain the plurality of glucose values.

12. The method of claim 11, comprising measuring the interstitial fluid glucose at irregular intervals over the period of time.

13. The method of claim 1, wherein the subject is non-diabetic or pre-diabetic.

14. The method of claim 1, wherein the subject is unmedicated for a metabolic disorder.

15. The method of claim 1, further including treating the subject for a metabolic disorder, metabolic imbalance or disturbance, or symptom thereof in response to a positive predicted insulin resistance.

16. A system for predicting insulin resistance in a subject, comprising:
a continuous glucose monitoring (CGM) sensor having an analyzer configured to measure glucose data from interstitial fluid of the subject and a transmitter configured to transmit the glucose data; and
a computation device comprising a receiver configured to receive the glucose data, the computation device being programmed to fit the measured glucose data from the subject to a glucose-insulin response model to calculate a plurality of subject-specific parameters and compare at least one of the calculated subject-specific parameters to a set threshold value to predict insulin resistance in the subject, the glucose-insulin response model comprising a modified Lotka-Volterra system of equations for the glucose-insulin response pair (G(t), I(t)).

17. The system of claim 16, wherein the computation device is further programmed to calibrate the predicted insulin resistance against a HOMA-IR homeostatic model assessment of insulin resistance to confirm the prediction of insulin resistance in the subject.

18. The system of claim 16, wherein the computation device is further programmed to generate the glucose-insulin response model from an initial measurement of glucose (G(t₀)) and an arbitrarily chosen value for insulin (I₀).

19. The system of claim 16, wherein the CGM sensor comprises a power source capable of powering the CGM sensor for at least 2 weeks.
